(19) United States
(12) Patent Application Publication
(10) Pub. No.: US 2025/0205032 A1
(43) Pub. Date: Jun. 26, 2025

(54) AN ANATOMICAL DEFECT REPAIR MESH

(71) Applicant: PerQHern Limited, Dublin (IE)

(72) Inventor: David KEANE, Dublin (IE)

(21) Appl. No.: 18/848,214

(22) PCT Filed: Mar. 20, 2023
(86) PCT No.: PCT/EP2023/057099

§ 371 Date: Sep. 18, 2024

(30) Foreign Application Priority Data
Mar. 21, 2022 (IE) 2022/0050

(51) Int. Cl. A61F 2/00

(52) U.S. Cl. CPC A61F 2/0063; A61F 2002/0068; A61F 2230/0006; A61F 2250/0003; A61F 2250/0013

(57) ABSTRACT

The present invention provides an anatomical defect repair mesh (10) having particular application in hernia repair, the mesh comprising a flexible substrate displaceable between a collapsed state for percutaneous delivery and an expanded state for overlying the anatomical defect and defining an array of substantially radially extending spokes (18), and an array of bridging struts (22) each provided between an adjacent pair of the spokes and extending obliquely thereto, the substrate preferably being cut from a single sheet of flexible material.

### FIELD OF THE INVENTION

This invention relates to a mesh for repairing an anatomical defect such an inguinal hernia, non-inguinal abdominal hernias (e.g. umbilical, ventral, incisional) cardiac and/or vascular defects or damage or the like, and in particular a repair mesh configured for percutaneous delivery to the site of the anatomical defect.

### BACKGROUND OF THE INVENTION

Prosthetic meshes for repairing anatomical defects such as hernias, muscle and vascular damage or defects are well known, a first generation of these meshes being generally composed entirely of polypropylene (PP). Although non-absorbable PP meshes are thought to have superior benefit over a simple suture such as used in the Bassini repair technique, initially these meshes were manufactured using small pore sizes and hence classified as “heavyweight” meshes. However, these properties promoted persistent inflammation and fibrosis and often significant mesh shrinkage. To address these issues meshes were developed composed of monofilament yarns of different thicknesses running parallel to each other. Further modifications involved anisotropic reinforcement with thicker yarns providing ideal stiffness and proper material unfurling, with higher number of thinner inner yarns to achieve mesh weight reduction and thus minimizing the amount of foreign material implanted.

However, a limited capability of withstanding biaxial tension, resulting in poor outcomes, was reported in clinical studies with a follow-up time of up to five to ten years. Mesh-related adhesion, erosion and implant fixation have also been reported as complications, especially for PP based meshes. Despite their limitations and high complication rates, the non-degradable surgical meshes are still marketed worldwide. Examples include PP meshes such as Marlex™, Prolene™, Prolite™, Atrium™, Trelex™, Surgipro™, meshes owned by TransEasyMedical Tech. Co. and expanded polytetrafluoroethylene (e-PTFE) meshes such as Gore-Tex™ and Mycromesh™.

Also made of non-degradable biomaterials such as medical grade PP, polyester, and e-PTFE, the second generation of surgical mesh aimed to reduce the inflammatory response, shrinkage, and the high rate of mesh adhesion associated with the first generation of synthetic meshes. To address these issues, PP meshes were manufactured in a lighter weight weave, using a combination of synthetic materials and mixtures thereof, as well as using therapeutic agent surface coating.

Prosthetic meshes classified as “lightweight” have been disclosed having claimed advantages including facilitation of fibroblast growth, promotion of macrophage and leukocyte ingrowth, reduction of chronic abdominal wall pain and foreign body sensation, biocompatibility and reduced risk of chronic pain, as well as offering similar anisotropic characteristics to that of abdominal tissues while having enough mechanical strength to prevent mesh rupture. Mainly, these prosthetic meshes are composed of a combination of PP and PTFE.

A major adverse event following hernia repair with prosthetic meshes is the adhesion of the mesh to internal organs. The introduction of a foreign body tends to initiate an acute inflammatory response, which promotes the deposition of fibrin and eventually adhesion of the material to the tissues of the organs. Disclosed solutions include an anti-blocking hernia patch featuring a PP mesh combined with chitosan and meshes with an anti-adhesion layer to prevent mesh adhesion, excellent repairing strength, reduced hernia recurrence rates, and reduced complication after surgery. In addition, meshes made of unique antiadhesive and coating layers. Coating materials incorporated to the prosthetic meshes included the use of hyaluronic acid, nitinol, titanium, hyaluronate carboxymethylcellulose hydrogel, oxidized regenerated cellulose, and chitosan.

Some surgical mesh have been disclosed which utilise nitinol in their composition, and for example feature a nitinol-reinforced lightweight PP mesh or a woven mesh made of PP, PTFE, or polyester with a shape memory alloy frame using nickel and titanium. The shape memory alloy frame allows the mesh to roll at cold temperatures and transforms back to planar form at 37° C., which facilitates easy insertion through laparoscopic surgery. Preclinical studies evaluating the use of nitinol in self-expanding meshes for laparoscopic hernia repair have been reported in the literature. Advantages claimed include mesh flexibility, thus preventing folds and shrinkage. It may also eliminate the need for mesh fixation, hence preventing chronic pain.

One of the most challenging complications of prosthetic meshes used for hernia repair is biofilm development and subsequent bacterial infection. Infection and biofilm formation on the mesh surface can severely hinder the repair process and in certain cases lead to additional surgeries, which increase the morbidity and mortality rates. These problems could potentially be addressed with mesh designs consisting of antibacterial and antifungal agents.

Derived from allogenic and xenogeneic sources, so called third generation surgical meshes were based on biological materials such as acellular collagen matrix scaffolds with or without cross-linking, to promote angiogenesis and tissue remodelling, while providing mechanical support. These biological meshes were introduced to improve the integration of the implant with the host tissue by facilitating vascularization and new tissue formation. Many were designed to be used in infected or potentially infected fields, where their biodegradable properties would reduce long term septic complications. However, accelerated rates of degradation and resulting collagen matrix formation are seen after the mesh implantation, leading to reduced elasticity and strength of the newly built tissue. Advantages over the first and second generation meshes include superior biocompatibility, reduced inflammatory responses, and promotion of wound healing responses over fibrosis formation. Drawbacks include high costs and poor biomechanical strength, leading to hernia recurrences.

Meshes have also been developed which incorporate an antibiotic coating into the prosthetic mesh to prevent mesh infection. Advantages claimed include biocompatibility, resorbability, and delivery of bioactive antibiotic agents. Despite other biomedical applications, the use of chitosan has also been reported in the art as part of a system for sustained antimicrobial drug delivery in combination with PP meshes. Positive results were obtained suggesting that the use of chitosan for controlled drug delivery could prevent mesh infection.

Partially absorbable and non-absorbable meshes are also available. Advantages of these meshes include decreased foreign body responses, reduced mesh adhesions, as well as the promotion of tissue ingrowth. One such prosthetic mesh includes a single plane tissue repair patch. The patch comprises a single layer and does not need a mesh anchoring or affixation layer. In addition, the patch has a bioabsorbable adhesion barrier attached to the bottom side. Improvements claimed include accelerating the rate of tissue integration, provision of less area for biofilm formation, less foreign body reaction, lower manufacturing costs, and easy to package, sterilize, and use with improved ergonomics. Adhesions, however, do still occur.

It is an object of the present invention to provide an improved repair mesh for an anatomical defect such as an inguinal hernia, muscle and/or vascular damage or defects which addresses some of the above mentioned problems of the prior art.

### SUMMARY OF THE INVENTION

According to a first aspect of the present invention there is provided an anatomical defect repair mesh comprising a flexible substrate displaceable between a collapsed state for percutaneous delivery and an expanded state for overlying the anatomical defect; wherein the substrate comprises an array of substantially radially extending spokes, and an array of bridging struts each provided between an adjacent pair of the spokes and extending obliquely thereto for at least a portion of the length of the bridging strut.

Preferably, at least some of the bridging struts extend obliquely along the full length of the strut.

Preferably, the substrate comprises an array of substantially circumferentially extending transverse members each extending between an adjacent pair of the spokes.

Preferably, one or more adjacent bridging struts and one or more adjacent transverse members meet at a common point on a respective spoke.

Preferably, the density of the spokes, bridging struts and/or transverse members varies across the substrate.

Preferably, the density of the spokes, bridging struts and/or transverse members varies such that an area of any individual space formed between said spokes, bridging struts and/or transverse members does not exceed a defined size.

Preferably, one or more of the radially extending spokes vary or are variable in stiffness along the length thereof.

Preferably, the substrate is non woven.

Preferably, the substrate is of unitary construction.

Preferably, the substrate is additive manufactured.

Preferably, the substrate is cut from a single sheet.

Preferably, comprising one or more expanding elements secured to or formed integrally with the substrate.

Preferably, the one or more expanding elements are removably coupled to the substrate.

Preferably, the one or more expanding elements are translatable between a collapsed state and an expanded state.

Preferably, the one or more expanding elements are resiliently deformable.

Preferably, the one or more expanding elements comprise a bladder adapted to receive and retain an inflating fluid in order to retain the substrate in the expanded state.

Preferably, the repair mesh comprises a circular array of radially extending bladders.

Preferably, the array of bladders are in fluid communication.

Preferably, the repair mesh comprises an externally accessible valve in fluid communication with the bladder.

Preferably, the repair mesh comprises an umbilical conduit connected between the valve and the bladder.

Preferably, the repair mesh comprises a closure operable to seal the umbilical to enable removal of the valve.

Preferably, the repair mesh comprises one or more guideways within which the one or more expanding elements are retained.

Preferably, each guideway comprise a sleeve provided on or in the substrate.

Preferably, the substrate comprises two or more planar layers overlying one another.

Preferably, the one or more expanding elements are defined by one or move voids formed between two of the planar layers.

Preferably, the sleeve comprises an enlarged peripheral end.

Preferably, one or more of the spokes comprise one or more bifurcations.

Preferably, the substrate is convex when in the expanded state.

Preferably, the repair mesh comprises one or more tensile reinforcing element extending across a concave face of the substrate with only opposed ends of the reinforcing element being secured to the substrate.

Preferably, the substrate is reversibly displaceable between the collapsed and expanded states.

Preferably, one or more of the spokes comprises one or more articulations along the length thereof separating the spoke into two or more articulated sections.

Preferably, the substrate comprises a centre region and an outer edge circumscribing the centre region, the spokes being arranged in a circular array around and extending radially outwardly from the centre region towards the outer edge.

Preferably, the repair mesh comprises an anchor operable to tether the substrate to tissue.

Preferably, the substrate defines a radial array of pleated sections defined by the spokes with adjacent pleated sections being foldable against one another to facilitate displacement of the substrate between the collapsed state and the expanded state.

Preferably, the substrate comprises a polymer.

Preferably, the substrate comprises polytetrafluorethylene.

Preferably, the repair mesh comprises one or more sensors on or about the substrate.

Preferably, the repair mesh comprises a port along a periphery of the substrate to accommodate an anatomical feature extending from the site of the anatomical defect.

As used herein, the term “transverse” is intended to mean that an element or structural member extends in a generally perpendicular direction relative to another element.

As used herein, the term “oblique” is intended to mean that an element or structural member is angled relative to another reference element, for example extending at an acute angle relative to the other element, as opposed to extending perpendicularly to the reference element.

### BRIEF DESCRIPTION OF THE INVENTION

The present invention will now be described with reference to the accompanying drawings, in which:

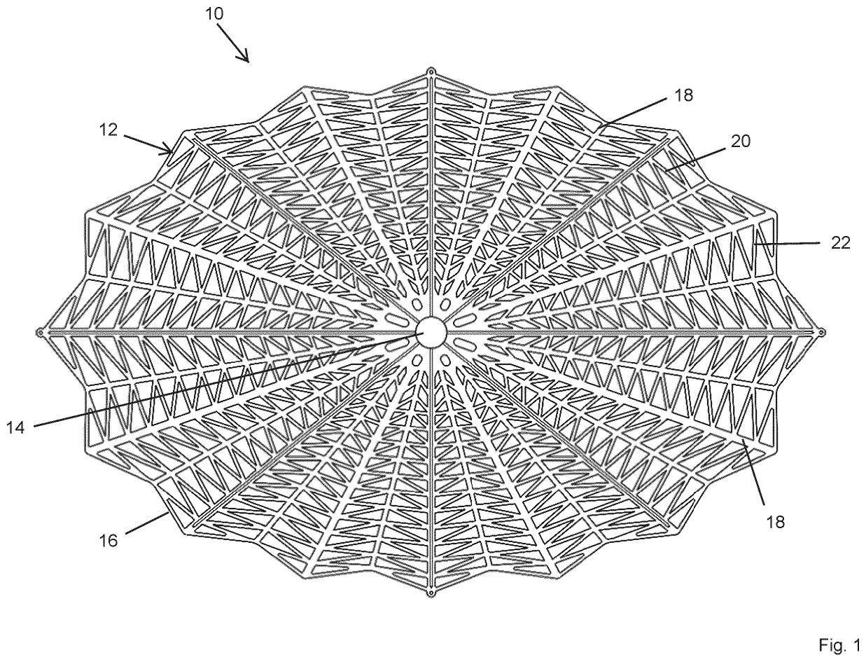

FIG. 1 illustrates a plan view of a repair mesh for an anatomical defect according to a first embodiment of the present invention;

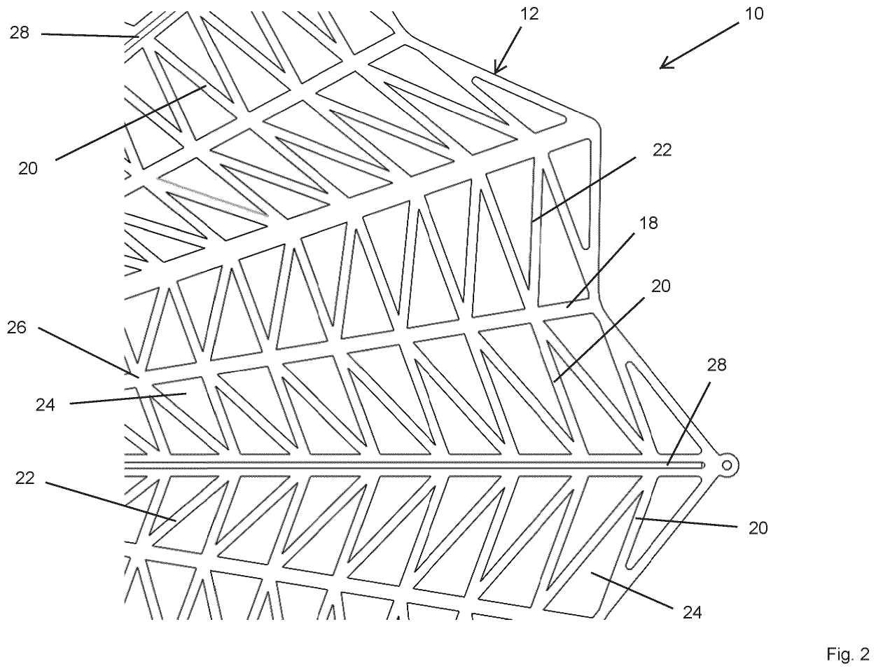

FIG. 2 illustrates an enlarged view of portion of the repair mesh shown in FIG. 1;

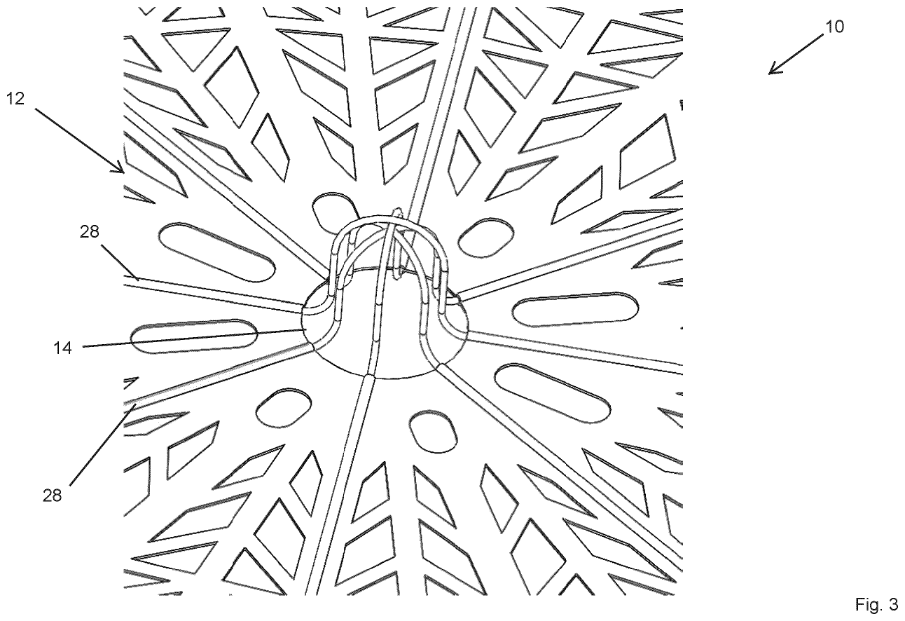

FIG. 3 illustrates an enlarged view from above of a centre portion of the repair mesh of FIG. 1 with a possible configuration for the central overlapping of a number of expanding members forming part of the repair mesh;

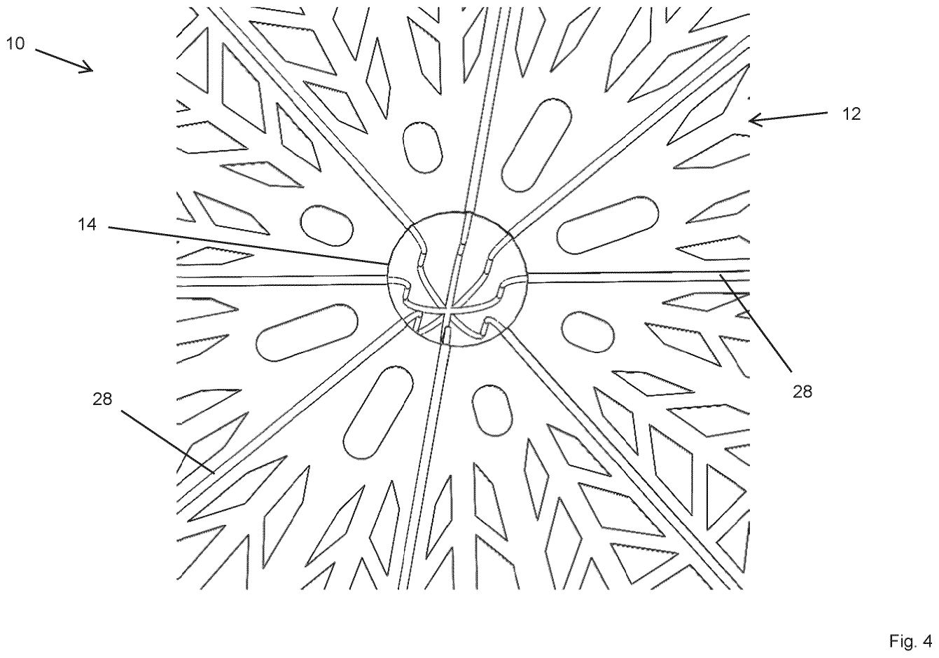

FIG. 4 illustrates the arrangement of expanding members at the centre region of the repair mesh as seen from an underside of the mesh;

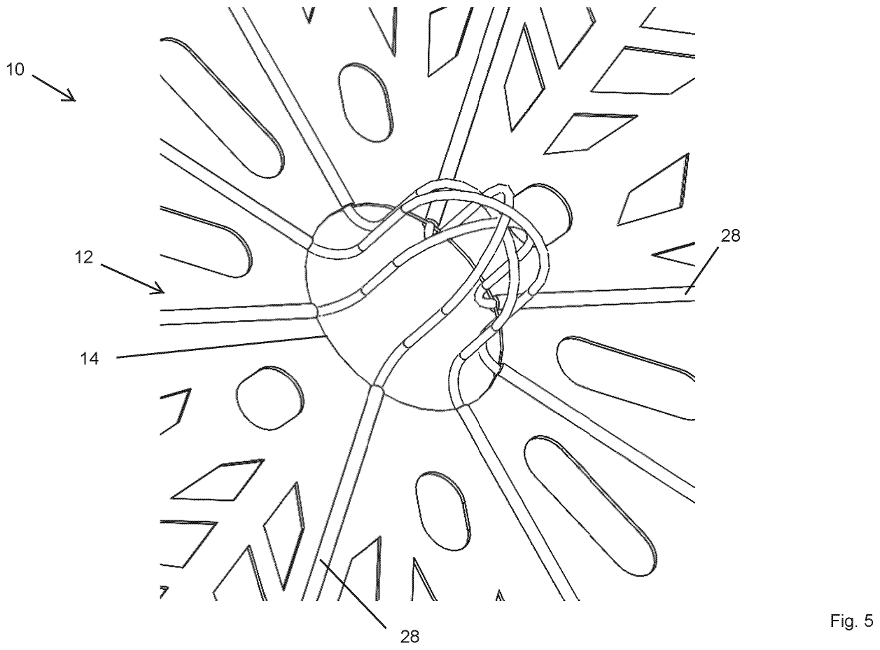

FIG. 5 illustrates an alternative perspective view of the arrangement shown in FIG. 3;

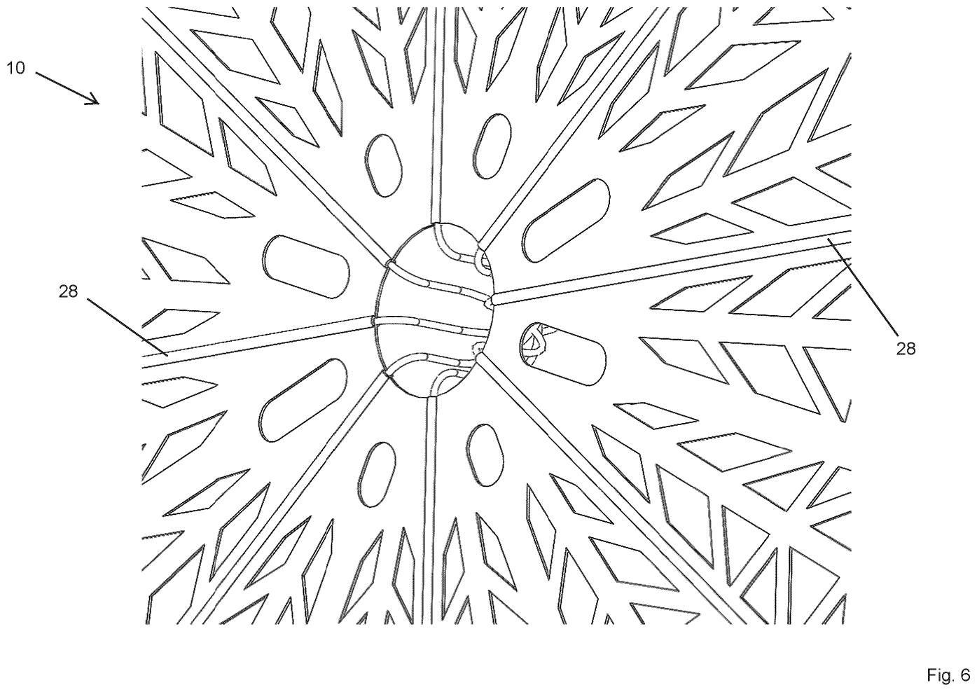

FIG. 6 illustrates an alternative perspective view of the arrangement shown in FIG. 4;

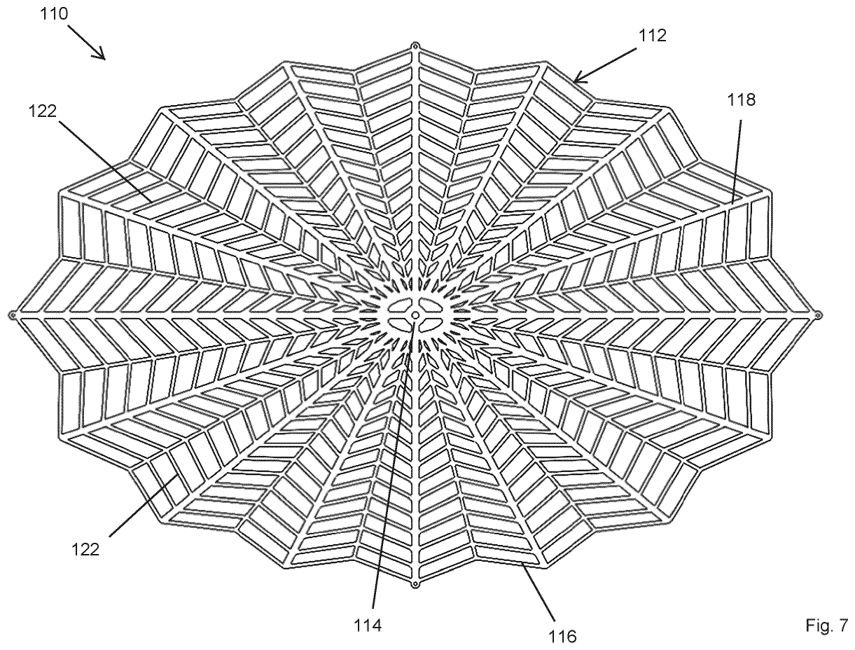

FIG. 7 illustrates a plan view of a repair mesh for an anatomical defect according to an altemative second embodiment of the present invention; and

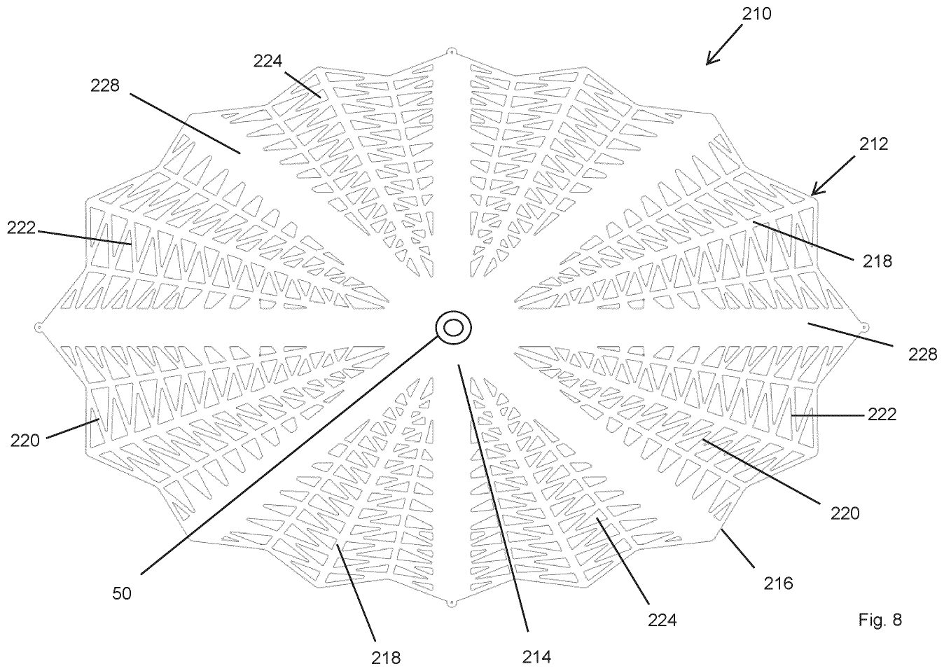

FIG. 8 illustrates a plan view of a repair mesh for an anatomical defect according to a third embodiment of the present invention.

### DETAILED DESCRIPTION OF THE INVENTION

Referring now to FIGS. 1 to 6 of the accompanying drawings there is illustrated a repair mesh for an anatomical defect such as an inguinal hernia, generally indicated as 10, according to a first embodiment of the present invention. The repair mesh 10 may be deployed and secured at various anatomical sites to serve as a barrier or reinforcement to damaged tissue, but is particularly intended for use in hernia repair. Furthermore the repair mesh 10 is adapted, as described in detail hereinafter, to be deployed in a minimally invasive manner such as using laparoscopic or percutaneous techniques, for example as disclosed in Applicant's earlier US patent U.S. Pat. No. 9,510,926B2 the details of which are incorporated herein. Such means of delivery and deployment are well known in the art and no further explanation thereof is deemed necessary for a comprehensive understanding of the configuration and operation of the present invention.

The repair mesh 10 is therefore displaceable between the expanded state as shown and a collapsed state for deployment to the anatomical site, in order to temporarily reduce the cross sectional area of the mesh 10 to facilitate the above mentioned minimally invasive deployment techniques. Once delivered to the anatomical site the mesh 10 may be displaced, as described hereinafter, into the expanded or deployed state as illustrated, to be secured over the anatomical defect in any suitable manner to provide reinforcement thereto. The mesh 10 thus comprises a flexible substrate 12 which is preferably circular or elliptical in shape, but other suitable shapes may also be employed to facilitate conformability to a patient's anatomical contours. The substrate 12 defines a centre region 14 and an outer edge or periphery 16 circumscribing the centre region 14. In use the substrate 12 may be collapsed into a generally conical or cylindrical arrangement with the centre region 14 forming an apex of the cone or one end of the cylinder and the outer edge 16 being compressed to form a base of the cone or other end of the cylinder to facilitate delivery, for example through a percutaneous sheath (not shown) or the like.

The substrate 12 further comprises a plurality of spokes 18 formed integrally therewith and arranged in a circular array radiating outward from at or adjacent the centre region 14 to at or adjacent the outer edge 16 of the substrate 12. The length of the spokes 18 may vary including but not limited to an alternating pattern where each pair of adjacent spokes 18 contains one short and one longer spoke 18 in alternation (i.e. every second spoke is longer). Similarly in the case of an elliptical mesh the spokes on and adjacent the major axis would be greater in length that the spokes on and adjacent the minor axis.

The substrate 12 additionally comprises a plurality of transverse members 20 formed integrally therewith and extending between adjacent spokes 18, along with a plurality of bridging struts 22 formed integrally with the substrate 12 and also extending between adjacent spokes 18 in a direction obliquely to the transverse members 20. Openings or voids 24 are therefore formed between spokes 18, transverse members 20 and bridging struts 22 which, in use, facilitate tissue ingrowth and thus increased anchoring of the repair mesh 10 to the anatomical site.

In a particularly preferred embodiment the entire substrate 12 is formed as a single monolithic or unitary structure, including the spokes 18, transverse members 20 and bridging struts 22. For example the substrate 12 may be laser cut or otherwise formed from a single sheet (not shown) of material such as a sheet of polytetrafluoroethylene or other suitable polymer or polymer composition. The voids 24 are therefore cut from the sheet to leave the interconnected arrangement of spokes 18, transverse members 20 and bridging struts 22 forming the substrate 12. The thickness, stiffness and/or other physical and compositional characteristics of the material may be varied to provide desired mechanical and operational properties to the mesh 10. Alternatively the substrate may be composed of two overlaid sheets (not shown). Alternatively the substrate 12 may be formed by additive manufacturing such as 3D printing to again provide a monolithic or unitary construction. By utilising such manufacturing techniques it is possible to accurately tune the physical configuration of the substrate 12 such as to produce particular mechanical characteristics which are not generally possible with conventional woven meshes, in addition to achieving a substrate 12 having a uniform thickness or accurately tuned variable thickness as required.

The number and/or dimensions of the structural elements of the substrate 12, in particular the spokes 18, transverse members 20 and bridging struts 22 may be varied as required, again to achieve desired physical or operational characteristics, and may be varied between different regions of the substrate 12. For example the substrate 12 may have a greater density of the structural elements around the central region 14 and a relatively lesser density or number of the structural elements towards the outer edge 16. In this way the physical characteristics of the substrate 12 can be tuned, for example to have a greater mechanical strength about the centre region 14 and a relatively lower mechanical strength, and therefore increased flexibility, towards the outer edge 16 and increased facility, opportunity and/or area or platform for tissue in-growth and adhesion and subsequent greater resistance to centripetal force on the mesh 10 and similarly through the bridging struts 22 to rotational force on the mesh 10. In general the mesh 10 is intended to be deployed such as to be substantially centred over the anatomical defect and it can therefore be beneficial that the centre region 14 has the greatest mechanical strength and thus provide the greatest support to the site of greatest anatomical weakness or damage. One or more dimensions of the spokes 18, transverse members 20 and bridging struts 22 may be varied in different regions of the substrate. For example the spokes 18 may decrease in width between the centre region 14 and the outer edge 18 to again provide increased flexibility of the mesh 10 in the peripheral region thereof. This can reduce focal pressure points and improve compliance with the local anatomy of the patient. Additional or alternative configurations may be employed to achieve the desired mechanical properties for the mesh 10. For example the one or more of the spokes 18 may have one or multiple bifurcations (not shown) as the spoke 18 extends radially towards the outer edge, branching into two or more sections.

Referring in particular to FIG. 2, in the preferred embodiment illustrated the substrate is laser cut from a single sheet of polytetrafluorethylene to define a circular array of thirty two spokes 18. Between pairs of adjacent spokes 18 extend a plurality of the transverse members 20, each therefore extending substantially perpendicularly to the connected spokes 18, which may also be defined as extending substantially tangential to the point of connection with the respective spoke 18. The radial distance between adjacent transverse members 20 may be constant or may vary, for example reducing towards the outer edge 16 in order to ensure the area of every void 24 does not exceed a certain predefined size regardless of the location of the void 24 on the substrate 12. Also extending between each pair of adjacent spokes 18 are a plurality of the bridging struts 22, each of which is linear and disposed obliquely to the spokes 18. In particular each bridging strut 22 extends outwardly from a point of connection between one of the spokes 18 and one of the transverse members 20 to a point of connection between the adjacent spoke 18 and the radially adjacent transverse member 20. Thus each local grouping of spoke 18, transvers member 20 and bridging strut 22 converge at single points or node 26. The voids 24 are therefore triangular in form as a result of this geometric arrangement of the structural elements of the substrate 12. The bridging struts 22 preferably extend at an acute angle to the adjacent transverse member 20, for example at an angle of between 30° and 70°, preferably 45°. The bridging struts 22 are preferably rectilinear and thus extend obliquely relative to the adjacent transvers members 20 along the full length of the bridging strut 22. However it is also envisaged that the bridging struts 22 could be curved or arched between the adjacent spokes 18.

The integral formation of the components or limbs forming the substrate 12 additionally provide improved resistance to rolling up or distortion of the mesh 10 over time and exposure to mechanical and tissue generated forces.

In order to facilitate unfurling of the substrate 12 into the expanded state once delivered to the site of the anatomical defect the mesh 10 preferably comprises one or more expanding elements, which in the preferred embodiment are in the form of resiliently deformable ribs 28, and which preferably extend radially or diametrically across the substrate 12. In a particularly preferred embodiment, referring to FIGS. 3 to 6, the ribs 28 are formed from a shape memory material such as nitinol, but any other suitable alternative may be employed, and multiple ribs 28 extend diametrically across the substrate 12, converging and passing one another at the centre region 14. The ribs 28 are spring biased to return to the straightened configuration shown, and thus when the mesh 10 is collapsed for delivery the ribs 28 will have to be forcibly deformed against the action of this spring biasing, most preferably by folding each diametrically extending rib 28 in half about the centre region 14. It will therefore be appreciated that once the substrate 12 is deployed from the respective delivery mechanism such as a percutaneous sheath (not shown) the ribs 28 will return to the straightened configuration and will thus act to unfurl the substrate 12 into the expanded state for fixation about the anatomical defect. In a particularly preferred embodiment there are four ribs 28 evenly spaced from one another at 0°, 90° 180° & 270°. The ribs 28 may have radio-opaque markers (not shown) thereon, preferably on a peripheral tip to allow location of the mesh 10 during deployment with suitable imaging equipment. The number and arrangement of the ribs 28 may of course vary.

The ribs 28 pass one another at the centre region 14 but are preferably not connected to one another. In a particularly preferred arrangement as illustrated each rib 28 may have a loop or corresponding feature at the midpoint therefore in order to create a projection at the centre region 14 which will, once the mesh 10 has been deployed and captured or sandwiched by the adjacent tissue opposing the anatomical defect such as between the peritoneum and bowel and the opposing inferior abdominal wall, will effectively cause the centre region 14 to be pressed into the anatomical defect, resulting in the mesh 10 taking on a convex form which will increase the load bearing capacity of the mesh 10 and thus greater resistance to downward or outward force form the anatomical defect, such as from the abdominal contents in the case of an inguinal hernia. It will be appreciated that any other functional alterative may be employed to establish this convex form in use.

The ribs 28 may be secured to the substrate 12 by any suitable means. For example the mesh 10 may comprise a radially/diametrically extending sleeve (not shown) secured to or formed integrally with one face of the substrate 12 and into which sleeve a respective rib 28 may be located. The ribs 28 may be permanently located in the sleeve and thus remain as part of the implanted mesh 10. In such a configuration the mesh 10 may be designed such that the ribs 28 form a structural component, providing a degree of mechanical support to the substrate 12, which may therefore be appropriated modified. In order to reduce contact point pressure at the peripheral tips of the ribs 28 it is preferred that the ribs 28 do not extend to the absolute peripheral or other edge 16 of the substrate 12.

Alternatively the ribs 28 may be releasably retained on the substrate 12, for example in the above mentioned sleeves or pockets (not shown) or otherwise, and may be separated from the substrate 12 following deployment at the site of the anatomical defect, to be appropriately removed. This may be done before or after the mesh 10 is secured to the site of the anatomical defect, for example using sutures, staples, adhesive or other appropriate means but the mesh 10 may not necessarily be secured routinely by such fixtures. This is particularly the case when a central tether and anchor (not shown) is incorporated into the mesh 10. Where the mesh 10 comprises sleeves (not shown) for retaining the ribs 28 the sleeves may be provided with an enlarged opening at one end of the sleeve in order to assist in removal of the respective rib 28 following deployment of the mesh 10 at the site of the anatomical defect.

It will be understood that functional alternatives to the ribs 28 may be employed in order to effect displacement of the mesh 10 from the collapsed to the expanded state. For example one or more sealed chambers, bladders or sleeves may be provided on or formed integrally with the substrate 12 as described hereinafter in relation a further embodiment shown in FIG. 8, preferably extending diametrically, and which may be inflatable with a liquid or gas, in order to force the mesh 10 from the collapsed state to the expanded state. The inflating liquid or gas may be supplied from a pre-filled canister (not shown) or the like. The inflating liquid (e.g. normal saline) or gas (e.g. CO_{2,} helium or air) may be allowed to passively deflate or may be actively extracted or otherwise removed once the mesh 10 has been deployed about the anatomical defect. However it is also envisaged that the inflating liquid or gas may be left in place by incorporation of a valve, which may provide the substrate with additional mechanical strength. The sleeves may be allowed or configured to deflate immediately or actively, or alternatively to deflate over a prolonged or tuned period of time. For example the sleeves (not shown) may remain inflated and supportive for a sufficient period of time to allow tissue ingrowth and therefore stabilisation of the mesh 10, and the sleeves may then slowly deflate, reducing rigidity and support and thus permitting tissue ingrowth to assume the support function. This gradual loss of rigidity will prevent pressure point pain or tenderness. The sleeves may be rolled up or otherwise compressed when the mesh 10 is in the collapsed state, and following delivery to the anatomical site the sleeves may be unrolled though inflation in order to expand the mesh 10 and provide support and structure thereto. A curable liquid may be used as the inflating liquid and which hardens following displacement of the mesh 10 into the expanded state. A liquid may be used which contains a radiographic dye or the like to allow imaging of the mesh 10 during the deployment procedure.

An alternative mechanism to achieve temporary stability of the mesh 10 for the initial period of deployment, for example the first month or two before the mesh 10 becomes adherent through tissue in-growth, is the use of magnesium for the ribs 28 or the provision of alternative magnesium struts (not shown) or the like. It is well recognized and established that magnesium has a highly predicable rate of dissolution in saline and in human solutions and that within a couple of months the magnesium ribs or struts would be fully dissolved and resorbed. The time to resorption of the magnesium ribs is determined by the thickness of the ribs, preferably contained within an open perforated sleeve (not shown) on the substrate 12 to allow body fluid to be exposed to the ribs or struts. This would further obviate the requirement for tacking or fixing the mesh 10 with staples or glue at the time of implant. Once resorbed the magnesium ribs or struts would avoid any potential for long-term pressure points and pain. It will of course be understood that any other suitable material may be selected, including but not limited to suitable polymers such polytetrafluorethylene (PTFE).

The mesh 10 may be provided with an anchor (not shown) for tethering the substrate 12 in position, for example as disclosed in Applicant's earlier U.S. Pat. No. 9,510,926B2. Such an anchor cord is operable to keep the centre region 14 of the mesh 10 located over the deep inguinal ring during inguinal hernia repair, and prevents migration of the mesh 10. The anchor (not shown) may be of any suitable configuration, for example the centre region 14 may have a tether or anchor point formed from a high tensile strength tether or cord (not shown) preventing the mesh 10 from migrating away from the hernia or abdominal weakness. A bar or tab (not shown) may be located at the free end of the tether defining an anchor to the tether the mesh 10 to a point outside the external inguinal ring (in the subcutaneous fat). The bar or tab can be made of permanent material which can be rigid or flexible (e.g. nitinol or PTFE) or can be a biodegradable material (e.g. magnesium or bio ceramics based on calcium phosphates or polylacticacid, etc.).

Referring now to FIG. 7 there is illustrated a repair mesh for an anatomical defect such as an inguinal hernia, generally indicated as 110, according to an alternative embodiment of the present invention. In this alternative embodiment like components have been accorded like reference numerals and unless otherwise stated perform like functions.

In this embodiment the mesh 110 comprises a substrate 112 which is again formed as a single monolithic or unitary part, preferably laser cut from a sheet of suitable material such as PTFE, or produced by any other suitable means. The substrate 112 defines a centre region 114, outer edge 116, and an array of radially extending spokes 118 therebetween. Extending between adjacent spokes 118 are a plurality of obliquely oriented bridging struts 122. The mesh 110 does not incorporate any transverse members as in the mesh 10 of the previous embodiment. However the substrate 112 can again be seen to have a greater density of material towards the centre region 114 with greater distance between the spokes 118 and bridging struts 122 peripherally. As detailed above this will ensure that the centre region 114 has a higher resistance to deformation and thus a greater load bearing capacity. The mesh 110 may again be provided with suitable expanding means (not shown) such as the shape memory ribs of the first embodiment, although any other suitable functional alternative may be employed.

Referring to FIG. 8 there is illustrated a repair mesh for an anatomical defect such as an inguinal hernia, muscle or vascular defects or damage, generally indicated as 210, according to a third embodiment of the present invention. In this third embodiment like components have been accorded like reference numerals and unless otherwise stated perform like functions.

The mesh 210 comprises a substrate 212 which is again constituted by a single monolithic or unitary part, preferably formed by two or more laminated layers each of which is laser cut from a sheet of suitable material such as PTFE, the two or more layers being suitable secured in face to face engagement as hereinafter described, preferably by means of heat welding or adhered together by any other suitable means. As with the previous embodiments the substrate 212 is planar in form and extends from a centre region 214 to an outer edge 216, comprising a circular array of spokes 218, the number of which may be varied as required or to suit a particular anatomical application. Between pairs of adjacent spokes 218 extend a plurality of the transverse members 220, extending substantially perpendicularly to the connected spokes 218 as hereinbefore described. The radial distance between adjacent transverse members 20 may be constant or may vary. Also extending between each pair of adjacent spokes 218 are a plurality of the bridging struts 222, each of which is linear and disposed obliquely to the spokes 218. Each bridging strut 22 preferably extends outwardly from a point of connection between one of the spokes 218 and one of the transverse members 220 to a point of connection between the adjacent spoke 218 and the radially adjacent transverse member 220. Voids 224 are formed therebetween and are triangular in form as a result of the geometric arrangement of the structural elements of the substrate 212. The bridging struts 222 preferably extend at an acute angle to the adjacent transverse member 220, for example at an angle of between 30° and 70°, more preferably at 45°.

In order to displace the substrate 212 between a collapsed state (not shown) and an expanded state as shown in FIG. 8, the mesh 210 comprises an array of expanding elements in the form of inflatable bladders 228 which preferably extend radially outwardly from at or adjacent a centre of the mesh 210. In the embodiment illustrated there are a circular array of radially extending bladders 228 which converge and are in fluid communication at the centre of the mesh 210, such that all the bladders 228 may be inflated/deflated simultaneously. It is however also envisaged that the bladders 228 may be individually inflated/deflated or may be inflated/deflated in groups. The mesh 210 comprises a port in the form of a valve 50 fixed to the substrate 212 and located at the centre of the mesh 210, the valve being in fluid communication with the interior of the bladders 228. Thus the valve 50 provides a means by which an inflating fluid may be introduced and/or removed from the bladders to displace the mesh 210 between the expanded and the collapsed state. The valve 50 may therefore be releasably connected to a suitable source of inflating liquid, for example a syringe with a manometer, gas canister, piped in-hosptial gas supplied (CO_{2,} O₂) etc. The valve 50 may for example be used to allow the bladders 228 to be serially inflated and deflated until deployment of the mesh 210 is considered to be optimal.

While the valve 50 is secured to the substrate 212 it is also envisaged that an umbilical type conduit (not shown) may be connected to the substrate in fluid communication with the bladders 228, with the valve 50 connected at the opposed free end of the conduit. This may improve access to the valve 50 during insertion of the mesh 210, and if left in situ may also allow the valve 50 to be positioned at an anatomically preferred location remote from the mesh or to use a larger valve 50 than would be possible or desirable when secured directly to the substrate. The use of an umbilical conduit and remote valve also allows the valve to be removed once the mesh 210 has been inflated, for example by simply cutting off the valve. In such a configuration a suitable closure (not shown) such as a band or clip or the like may be provided about the umbilical conduit and which may be tightened or otherwise clamped onto the conduit in order to close same prior to removal of the valve.

It is also envisaged that the valve could remain in place on the umbilical conduit again to allow the pressure within the bladders 228 to be adjusted post operatively in order fine tune the stiffness of the mesh 210. The valve may also function as the above described anchor in order to retain the mesh 210 in position over the anatomical site to be repaired. Alternatively the umbilical conduit could be branched to include a first leg on which the valve is provided and a parallel second leg containing the anchor.

The inflating liquid may be a fluid or gas, for example carbon dioxide, air (which is radiolucent) liquid and/or gel silicone, liquid and/or curable polymers, or any other suitable fluid, whether biocompatible and/or biodegradable or not. The bladders 228 are preferably fluid impermeable in order to permanently contain the inflating fluid, but it is also possible that a level of porosity may be provided in order to allow the bladders 228 to deflate over a period of time, thereby avoiding long term pressure points. The bladders 228 may also be inflatable to different pressures, or may be of different dimensions, in order to allow tuning of the stiffness of the mesh 210 when in the expanded state. The bladders 228 may also be provided as separate enclosures captured or contained within an outer sleeve (not shown) provided on or formed integrally with the substrate 212 in order to provide a space surrounding the bladders 228 into which any leaking inflating fluid may be retained in order to avoid contamination of the surround tissue, depending on the inflating fluid that is employed. This configuration may be achieved by providing the array of bladders 228 as a separate component which is then captured or sandwiched between two layers or laminates which are secured to one another with the bladders 228 therebetween to form the substrate 212. The two layers may be secured together with heat and/or pressure, thermally welded or otherwise secured face to face to one another, with the space immediately surrounding each bladder 228 being left unsecured in order to define the above mentioned space or sleeve surrounding the bladders 228. It will of course be understood that any other suitable means of manufacturing the mesh 210 in order to achieve such a physical configuration may be employed.

The mesh 10; 110; 210 may be pleated, for example into sections defined by the spokes 18; 118; 218 and which are foldable relative to one another about fold lines defined by the spokes 18; 118 in order to move the mesh 10; 110; 210 between the expanded and collapsed state, similar to the movement of the canopy of an umbrella. The mesh 10; 110; 210 may comprise one or more sensors (not shown) to monitor one or more parameters such monitoring pressure at the anatomical site, monitoring for biomarkers or other signs of disease or infection, or any other useful parameter or bio sign. Such sensors may include but are not limited to electronic detection of biochemical and metabolic parameters, physical parameters (temperature, tension), cardiovascular parameters and the transmission of the data wirelessly). The power for such sensors and data transmission may be derived by external wireless power or mechanical compression within the abdomen. The mesh 10; 110 may also include a second or further layers (not shown) partially or fully overlying the substrate 12; 112; 212.

The mesh 10; 110; 210 may be modified to include an opening or portal (not shown) in the centre of the mesh 10; 110; 210 to allow the passage of one or more surgical instruments during the deployment and/or removal of the mesh 10; 110; 210. For example some form of scaffold or support (not shown) may be passed through the portal in the mesh 10; 110; 210 prior to full deployment, which support may be utilised to create an operating space or cavity around the mesh 10; 110; 210 which may aid in the deployment and/or visualisation via fibre optic imaging or the like. The support may for example take the form of a deployable or inflatable canopy or the like which could be located directly adjacent/above the mesh 10; 110; 210 and which can be deployed to create the necessary space to facilitate improved/complete expansion of the mesh 10; 110; 210 from the collapsed state utilised to deliver the mesh 10; 110; 210 to the anatomical repair site. This canopy (not shown) may be an integral part of the mesh 10; 110; 210 or may be a separate component that may be retrieved through the portal (not shown) once the mesh 10; 110; 210 has been satisfactorily deployed.

As detailed above, the mesh 10; 110; 210 is preferably domed or convex in form in order to provide structural rigidity during operation and to prevent the mesh 10; 110; 210 from collapsing under pressure/movement, etc. In order to further improve this aspect the mesh 10; 110; 210 may comprise reinforcing elements such as one or more tensile members (not shown) or webbing extending across an underside (concave face) of the mesh 10; 110; 210, preferably extending diametrically and being dimensioned to be in tension when the mesh 10; 110; 210 is in the expanded state. These members will therefore act to resist the collapse or inversion of the mesh 10; 110; 210 when under load, for example to greater resist pressure from abdominal contents.

The design of the mesh 10; 110; 210 of the invention enables the above mentioned characteristics to be finely tuned, for example variable flexibility between the centre region 14; 114; 214 and the outer edge 16; 116; 216; enabling improved tissue adhesion; and resistance to movement and/or collapse following securement at the anatomical defect. The oblique orientation of the bridging struts 22; 122; 222, once embedded by tissue ingrowth, prevent the mesh 10; 110 from undergoing rotation. The transverse members 20; 120; 220 combined with the bridging struts 22; 122; 222, again once embedded by tissue ingrowth, prevent implosion of the mesh 10; 110; 210, in particular towards the centre region 14; 114; 214. In addition the oblique orientation of the bridging struts 22; 122; 222 in combination with the horizontal or transversely extending transverse members 20; 120; 220 provide maximal frictional resistance against inward migration (in-folding/implosion) of the mesh 10; 110; 210 and facilitate tissue in-growth and fastening of the mesh 10; 110; 210 at the site of the anatomical defect.

The repair mesh 10; 110 of the present invention therefore provides for improved mechanical characteristics to be accurately established and which may by design be varied across different regions of the mesh 10; 110 to provide improved performance.

The invention is not limited to the embodiments described herein but can be amended or modified without departing from the scope of the present invention.

1. An anatomical defect repair mesh comprising a flexible substrate displaceable between a collapsed state for percutaneous delivery and an expanded state for overlying the anatomical defect; wherein the substrate comprises an array of substantially radially extending spokes, and an array of bridging struts each provided between an adjacent pair of the spokes and extending obliquely thereto for at least a portion of the length of the bridging strut.

2. The repair mesh of claim 1 wherein at least some of the bridging struts extend obliquely along the full length of the strut.

3. The repair mesh of claim 1 wherein the substrate comprises an array of substantially circumferentially extending transverse members each extending between an adjacent pair of the spokes.

4. The repair mesh of claim 3 wherein one or more adjacent bridging struts and one or more adjacent transverse members meet at a common point on a respective spoke.

5. The repair mesh of claim 1 wherein the density of the spokes, bridging struts and/or transverse members varies across the substrate.

6. The repair mesh of claim 1 wherein two or more of the spokes are of different length.

7. The repair mesh of claim 5 wherein the density of the spokes, bridging struts and/or transverse members varies such that an area of any individual space formed between said spokes, bridging struts and/or transverse members does not exceed a defined size.

8. The repair mesh of claim 1 wherein one or more of the radially extending spokes vary or are variable in stiffness along the length thereof.

9. The repair mesh of claim 1 wherein the substrate is non woven.

10. The repair mesh of claim 1 wherein the substrate is of unitary construction.

11. The repair mesh of claim 1 wherein the substrate is additive manufactured.

12. The repair mesh of claim 1 wherein the substrate is cut from a single sheet.

13. The repair mesh of claim 1 comprising one or more expanding elements secured to or formed integrally with the substrate.

14. The repair mesh of claim 13 wherein the one or more expanding elements are removably coupled to the substrate.

15. The repair mesh of claim 13 wherein the one or more expanding elements are translatable between a collapsed state and an expanded state.

16. The repair mesh of claim 13 wherein the one or more expanding elements are resiliently deformable.

17. The repair mesh of claim 13 wherein the one or more expanding elements comprise a bladder adapted to receive and retain an inflating fluid in order to retain the substrate in the expanded state.

18. The repair mesh of claim 17 comprising a circular array of radially extending bladders.

19. The repair mesh of claim 18 in which the array of bladders are in fluid communication.

20. The repair mesh of claim 17 comprising an externally accessible valve in fluid communication with the bladder.

21. The repair mesh of claim 20 comprising an umbilical conduit connected between the valve and the bladder.

22. The repair mesh of claim 20 comprising a closure operable to seal the umbilical to enable removal of the valve.

23. The repair mesh of claim 13 comprising one or more guideways within which the one or more expanding elements are retained.

24. The repair mesh of claim 23 wherein each guideway comprise a sleeve provided on or in the substrate.

25. The repair mesh of claim 24 wherein the sleeve comprises an enlarged peripheral end.

26. The repair mesh of claim 13 wherein the substrate comprises two or more planar layers overlying one another.

27. The repair mesh of claim 26 wherein the one or more expanding elements are defined by one or move voids formed between two of the planar layers.

28. The repair mesh of claim 1 wherein one or more of the spokes comprise one or more bifurcations.

29. The repair mesh of claim 1 wherein the substrate is convex when in the expanded state.

30. The repair mesh of claim 27 comprising one or more tensile reinforcing element extending across a concave face of the substrate with only opposed ends of the reinforcing element being secured to the substrate.

31. The repair mesh of claim 1 wherein the substrate is reversibly displaceable between the collapsed and expanded states.

32. The repair mesh of claim 1 wherein one or more of the spokes comprises one or more articulations along the length thereof separating the spoke into two or more articulated sections.

33. The repair mesh of claim 1 wherein the substrate comprises a centre region and an outer edge circumscribing the centre region, the spokes being arranged in a circular array around and extending radially outwardly from the centre region towards the outer edge.

34. The repair mesh of claim 1 wherein the substrate defines a radial array of pleated sections defined by the spokes with adjacent pleated sections being foldable against one another to facilitate displacement of the substrate between the collapsed state and the expanded state.

35. The repair mesh of claim 1 comprising an anchor operable to tether the substrate to tissue.

36. The repair mesh of claim 1 wherein the substrate comprises a polymer.

37. The repair mesh of claim 1 wherein the substrate comprises polytetrafluorethylene.

38. The repair mesh of claim 1 comprising one or more sensors on or about the substrate.

39. The repair mesh of claim 1 comprising a port along a periphery of the substrate to accommodate an anatomical feature extending from the site of the anatomical defect.
